(19) United States
(12) Patent Application Publication
(10) Pub. No.: US 2003/0181375 A1
(43) Pub. Date: Sep. 25, 2003

(54) Rapid phenotypic cell-based HIV assay

(76) Inventor: James J. McSharry, Albany, NY (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/350,459

(22) Filed: Jan. 24, 2003

Related U.S. Application Data
(60) Provisional application No. 60/351,789, filed on Jan. 25, 2002.

(51) Int. Cl. C12Q 1/70; A61K 38/16

(52) U.S. Cl. 514/12; 435/5

(57) ABSTRACT

Rapid phenotypic drug susceptibility assays that detect drug resistance in HIV clinical isolates irrespective of the genes containing mutations that lead to drug resistance. The invention provides assays for determining HIV infection and the degree thereof; for determining the efficacy of candidate HIV inhibitors; and for clinically monitoring the progress of HIV therapies. In one embodiment, methods of the invention feature infecting a cell line, expressing CD4, CXCR4 and CCR5 receptors on the cell surface, and a marker gene product, with HIV in the presence of a putative HIV inhibitor, wherein the marker gene product expression increases in response to said cell line infection with HIV; and dynamically counting the number of said cells, e.g., by flow cytometry, expressing the marker gene product and comparing the number of cells expressing the marker gene product to a control value to determine whether the putative HIV inhibitor is an inhibitor of HIV.

### RELATED APPLICATIONS

This application claims the benefit of priority under 35 U.S.C. 119(e) to copending U.S. Provisional Application No. 60/351,789 (19705-011 PRO), filed Jan. 25, 2002; the entire content of which is incorporated herein by reference.

### FIELD OF THE INVENTION

The invention relates to assays and methods for determining the infectivity and viral resistance/sensitivity of isolates of an immunodeficiency virus.

### BACKGROUND OF THE INVENTION

The invention relates to assays and methods for determining the infectivity and viral resistance/sensitivity of isolates of an immunodeficiency virus. The invention is useful for diagnosing HIV, the discovery of new drugs effective against HIV, and monitoring drug therapy protocols to enhance the effectiveness of drug treatment regimes against HIV, e.g., HIV-1 infection.

Treatment of human immunodeficiency virus (HIV) infected individuals with effective antiviral therapy has greatly reduced the mortality from AIDS, the disease caused by HIV. However, over the course of treatment with these antiviral agents there are many clinical failures. Most clinical failures are caused by the selection of HIV strains that are resistant to one or more of the antiretroviral agents used in therapy. These drug resistant HIV strains continue to replicate and cause disease even in the presence of antiviral drugs.

There are a number of genotypic and phenotypic assays available to determine the drug susceptibilities of HIV clinical isolates obtained from HIV infected patients who fail therapy. Genotypic assays detect mutations in the reverse transcriptase and the protease genes that may be related to drug failure. The phenotypic assays detect drug resistance irrespective of the mutations and are more useful for detecting drug resistance than the genotypic assays, but phenotypic assays are time consuming, labor intensive, and expensive. The homologous recombination phenotypic assays are more rapid, but as currently configured only detect drug resistance associated with mutations in the reverse transcriptase and the protease genes.

There is also currently no cost effective, “high throughput” method for analyzing the drug resistant phenotype of primary virus isolates derived from individuals receiving antiretroviral treatment, i.e., clinical isolates. Several assays have been published on techniques for determining drug susceptibilities of cell free HIV (Shannon et al., Abstracts of HIV DART 2000 Conference, Puerto Rico, Dec. 17-21, 2000, #76; Petropoulous et al., 2000, Antimicrob. Agents Chemother. 44:920-928; Pirounaki et al., 2000, J. Virol. Meth 85:151-161; Hachiya et al. 2001 Antimicrob. Agents Chemother. 45:495-501; Fleming et al., Abstracts of 14^{th} International Conference on Antiviral Research, April 2001, #36). These assays use, however, a 96 well format and measure outcome with a spectrofluorometric reader or count the number of blue cells (β-gal expressing cells) with the aid of a light microscope.

Various in vitro biologic and immunologic techniques have been developed to detect human and simian immunodeficiency viruses (HIV and SIV, respectively). These include assays that detect the enzymatic activity of the reverse transcriptase (RT) protein, ELISA based assays for the detection of HIV/SIV core antigen (HIV-I p24 or HIV-2/SIV p27), direct quantitation of infectious virus by syncytial focus plaque assays or limiting dilution titration in susceptible host cells, visualization of virusesons by electron microscopy, in situ hybridization, and various nucleic acid-based assays. Genetic reporter-based assays have also been created to detect HIV/SIV infection, wherein mammalian cells are genetically modified to express a reporter gene such as P-galactosidase, green fluorescent protein, or chloramphenicol acetyltransferase in response to infection and Tat protein expression. These detection systems require enumeration of the number of infection-positive cells, e.g., by fluorescence microscopy.

### SUMMARY OF THE INVENTION

The present invention relates in part to rapid phenotypic drug susceptibility assays that detect drug resistance in HIV clinical isolates irrespective of the genes containing mutations that lead to drug resistance. The invention provides assays for determining HIV infection and the degree thereof; for determining the efficacy of candidate HIV inhibitors; and for clinically monitoring the progress of HIV therapies. In one embodiment, methods of the invention feature infecting a cell line, expressing CD4, CXCR4 and CCR5 receptors on the cell surface, and a marker gene product, with HIV in the presence of a putative HIV inhibitor, wherein the marker gene product expression increases in response to said cell line infection with HIV; and dynamically counting the number of said cells, e.g., by flow cytometry, expressing the marker gene product and comparing the number of cells expressing the marker gene product to a control value to determine whether the putative HIV inhibitor is an inhibitor of HIV. Marker gene expression correlates with the magnitude of virus infection.

The marker gene product may desirably be a cell-associated marker composition such as a luminescent protein like GFP. The cell line may be, e.g., osteosarcoma cells, and which may further express other receptors such as CCR1, CCR2b, CCR3, CCR4, CCR8, CXCR1, CXCR2, CXCR3, CX, CR1, STRL22/BONZO or GPR15/BOB.

The invention further relates to methods for screening clinical isolates for determining the efficacy of candidate HIV inhibitors, featuring contacting a cell line of the invention with a clinical isolate suspected of containing HIV in the presence of a putative HIV inhibitor, and dynamically counting the number of cells expressing the marker gene product and comparing the number of cells expressing the marker gene product to a control value to determine whether the putative HIV inhibitor is an inhibitor of HIV.

### BRIEF DESCRIPTION OF THE DRAWING

FIG. 1 illustrates the detection of GFP expression in R3/X4/R5 cells by flow cytometry;

FIG. 2 shows an HIV drug susceptibility assay, for cell free viruses, in accordance with the invention; and

FIGS. 3 and 4 show the use of virus infected cells in a drug susceptibility assay of the invention.

### DETAILED DESCRIPTION OF THE INVENTION

The features and other details of the invention will now be more particularly described with reference to the accompanying drawings and pointed out in the claims. It will be understood that particular embodiments described herein are shown by way of illustration and not as limitations of the invention. The principal features of this invention can be employed in various embodiments without departing from the scope of the invention. All parts and percentages are by weight unless otherwise specified.

The invention provides assays for determining HIV infection and the degree thereof; for determining the efficacy of candidate HIV inhibitors; and for clinically monitoring the progress of HIV therapies. In one embodiment, methods of the invention feature infecting a cell line, expressing CD4, CXCR4 and CCR5 receptors on the cell surface, and a marker gene product, with HIV in the presence of a putative HIV inhibitor, wherein the marker gene product expression increases in response to the cell line infection with HIV; and dynamically counting the number of the cells, e.g., by flow cytometry, expressing the marker gene product and comparing the number of cells expressing the marker gene product to a control value to determine whether the putative HIV inhibitor is an inhibitor of HIV.

The methods of the invention are particularly useful and noteworthy as currently there are rather few good options for monitoring clinical cell specimens for the presence of HIV, since the level of detection of the viruses in vivo is low with current methods. The invention overcomes this obstacle by allowing for the analysis of both cell-free virus and chronically infected cells.

The methods of the invention can be used for clinical analysis, drug discovery, and diagnostic purposes. The methods of the invention are based, at least in part, on the recognition that the use of cell lines expressing at least CD4, CXCR4 and CCR5 and a marker gene product, (particularly a cell-associated marker gene product), with a dynamic counting to determine the numbers of infected cells, e.g., by flow cytometry, results in a faster and more sensitive and accurate determination than heretofore achievable.

Definitions

For convenience, certain terms used in the specification, examples, and appended claims are collected here.

“Candidate HIV inhibitor” or “putative HIV inhibitor” includes compounds or compositions that are reasoned or believed to be inhibitors of HIV.

“HIV” includes all known retroviruses belonging to the HIV group, e.g., HIV-1, HIV-2, and SIV, as well as those in existence but not yet formally recognized as such. These retroviruses give rise, in humans infected with them, to disease symptoms which are summarized under the collective term immune deficiency or AIDS (acquired immune deficiency syndrome). (Epidemiological studies demonstrate that the human immunodeficiency virus (HIV) represents the etiological agent for the overwhelming majority of AIDS cases.)

“Marker gene product” includes products produced by expression of nucleic acid sequences encoding proteins which may be assayed by various techniques. Exemplary marker gene products include chloramphenicol acetyltransferase (CAT); β-galactosidase (GAL); β-glucuronidase (GUS); luciferase (LUC); and green fluorescent protein (GFP).

“Dynamically counting” includes methods of cell counting, such as flow cytometry, which involve the use of dynamic, rather than static methods such as immunoassay in multiwell plates, or cell counting under a microscope.

“Cell-associated marker composition” includes marker gene products expressed by the cell line of the invention which remain associated with the cell, e.g., on, proximate to, or in the cell, allowing the cell to be more accurately counted using dynamic counting methods. An exemplary cell-associated marker composition is GFP.

“Luminescent protein” includes any protein known to those of ordinary skill in the art to provide a readily detectable source of light when present in stable form. Non-limiting examples include light-generating proteins described in U.S. Pat. Nos. 5,683,888, 5,958,713, and 5,650,135, e.g., ferredoxin IV, green fluorescent protein, red fluorescent protein, yellow fluorescent protein, blue fluorescent protein, the luciferase family and the aequorin family. In a preferred embodiment, the light-generating polypeptide moiety is a protein such as green fluorescent protein, red fluorescent protein, yellow fluorescent protein and blue fluorescent protein.

“Primary HIV” refers to HIV derived directly from an infected host organism from sources such as blood, plasma, and other tissues.

“Drug susceptibility” or “drug resistivity” alternately refers to the effectiveness of a drug to inhibit HIV replication and/or expression with a host cell.

The term “T-trophic virus” refers to a phenotype of an immunodeficiency virus capable of infecting a T-cell by binding the CD4 receptor on the T-cell.

“Macrophage trophic virus” includes a phenotype of an immunodeficiency virus capable of infecting a macrophage by binding the CCR5 co-receptor on the macrophage. This difference in tropism has been mapped to the viral env gene. The SG3 (Ghosh et al. 1993, Virology 194:858-864) and NL43 (Paxton et al. 1993, J. Virol. 67:7229-7237) strains of HIV-1 are derived by extensive passage in tissue culture. They represent T-cell tropic viruses and do not infect monocytes and macrophages. These viruses are not representative of the complex mixtures of viruses that exit in infected individuals. Primary HIV-1 represent viruses that are derived directly from the blood of an HIV infected individual. Primary HIV can also be derived by short term in vitro culture in primary peripheral blood mononuclear cells (PBMC). Such isolates are complex mixtures and may contain macrophage- and/or T-tropic viruses. During the natural history/progression of HIV-1 infection there is generally a shift from a population of macrophage-tropic toward one of T-tropic viruses. T-cell tropic viruses are able to infect cells that express CD4 and CXCR4, while macrophage tropic (M-tropic) viruses also require expression of the CCR5 chemokine co-receptor. Most HIV-2 and SIV viruses also require the CCR5. Several groups have produced cell lines that express CD4, CXCR4 and CCR5 in attempts to render them sensitive to infection with primary HIV-1 (both T-cell and macrophage tropic viruses). Only recently have such cell lines been derived which appear to be susceptible to infection with diverse HIV-1 isolates (Platt et al., J. Virol. 72:2855, 1988; Overbaugh et al., J. Virol. 71:3932, 1997).

The assays of the invention may be used for determining the susceptibilities of HIV laboratory strains and clinical isolates to the antiretroviral drugs currently used in the treatment of HIV infection. The assays determine drug susceptibilities of HIV irrespective of the mutations in genes associated with drug resistance. The assays are particularly useful for agents that affect the attachment, uncoating, reverse transcription and integration steps in the HIV replication cycle. A cell line in accordance with the invention includes the genetically engineered osteosarcoma cell line R3/X4/R5 (obtained through the NIH AIDS Research and Reference Reagent Program). R3/X4/R5 expresses CD4, CXCR4, and CCR5 on the cell surface and green fluorescent protein (GFP) when productively infected with HIV. In a preferred embodiment, the number of HIV infected cells expressing GFP is detected and quantified by flow cytometry. When the assay is performed in the presence of various concentrations of antiviral drugs, the percentage of cells expressing GFP decreases with increasing drug concentrations. The concentration of drug that reduces the number of cells expressing GFP by 50% (IC₅₀) may be determined by calculating the reduction in the percentage of GFP expressing cells at each drug concentration from the percentage of GFP expressing cells in the absence of drug and plotting the percent reduction against the drug concentration.

The assays of the invention may be used to determine the drug susceptibilities of a number of drug susceptible and drug resistant HIV laboratory strains and clinical isolates to nucleoside reverse transcriptase inhibitors including Zidovudine (AZT), Lamivudine (3TC), and Abacavir Succinate (ABC); non-nucleoside reverse transcriptase inhibitors including Efavirenz (EFA), Delavirdine (DLV), and protease inhibitors such as Amprenivir (AMP). The assay can be used to determine drug susceptibilities of lymphotropic, macrophage tropic, and dual tropic strains of HIV.

The current literature suggests that between 1% and 50% of the circulating CD4⁺ T cells in HIV infected patients contain HIV proviral DNA (Patterson et al., 1995, J. Virol 69:4316-4322; Haase, 1999, Ann. Rev. Immunol. 17:625-656). These are not the CD4⁺ memory T cells that are present in very small numbers, but the CD4⁺ T cells that are abortively infected with HIV and fail to induce a productive infection. These HIV infected CD4⁺ T cells have a half live of 6 to 8 days and are continually being produced as CD4⁺ T cells productively infected with HIV produce more virus (Ho, 1997, J. Clin. Investig. 99:2565-2567; Richman, 2001, Nature 410: 995-1001). If these reports are accurate, then 10⁸ to 10¹⁰ lymphocytes are available for analysis of drug susceptibilities of the virus contained within those CD4⁺ T cells.

The assays of the invention may be used to determine the susceptibility of HIV clinical isolates to the various therapies that are used to treat HIV infected patients, e.g., obtained directly from the peripheral blood of HIV infected individuals. The assays feature several distinctive characteristics: 1) the use of both cell free virus and HIV infected cells to determine drug susceptibilities of HIV clinical isolates, which avoids having to grow and quantify the virus before analysis of drug susceptibility; 2) the use of flow cytometry to detect and quantify the number of marker gene product, e.g., GFP, expressing cells; 3) the rapidity of the assay (generally 3 days for cell activation and 2 days for the assay); and 4) the ease and quantitative nature of the assay (each cell expressing marker gene product is counted). The assay may be completed in less than one week, a time frame that is more rapid than the homologous recombination assays currently used to monitor treatment for HIV infected patients.

### EXAMPLE 1

The following describes the assay and data showing the use of the assay to determine the concentration of drug that reduces the number of R3/X4/R5 cells that express green fluorescent protein (GFP) after infection with HIV by 50% (IC₅₀). Data are presented showing the effect of nucleoside reverse transcriptase inhibitors, non-nucleoside reverse transcriptase inhibitors, and a protease inhibitor on the replication of HIV laboratory strains and clinical isolates in the R3/X4/R5 cells. Data are also presented showing the use of HIV infected cells as the source of HIV in this assay.

Cells: R3/X4/R5 cells were obtained from the NIH AIDS Research and Reference Reagent Program, Division of AIDS, National Institutes of Allergy and Infectious Diseases, National Institutes of Health. The cells were maintained as monolayer cultures in Dulbecco's modified minimal essential medium supplemented with 10% heat inactivated fetal bovine serum, penicillin, streptomycin, G418, puromycin and hygromycin B.

Viruses: HIV clinical isolates were obtained from the Albany Medical College AIDS Clinical Trials Group (ACTG) and the NIH AIDS Research and Reference Reagent Program. The properties of the viruses are listed in Table 1. HIV clinical isolates were grown in PHA and IL-2 stimulated peripheral blood cells obtained from the Red Cross or in PM1 cells in RPMI 1640 medium with 10% heat inactivated fetal bovine serum (FBS), penicillin and streptomycin. HIV replication was monitored using a Coulter p24 Antigen Detection Kit. HIV was harvested when the p24 antigen in the supernatants of virus cultures reached 200 to 500 nanograms per ml. Virus stocks were clarified buy centrifugation at 1000 rpm for 10 min and the supernatants were stored at −70° C.

Antiviral Drugs: Zidovudine (AZT), abacavir succinate (ABC), and efavirenz (EFV) were obtained from GlaxoSmithkline, Lamivudine (3TC) was obtained from Bristol Myers Squibb, and Amprenavir (AMP) was obtained from Pfizer Global.

The Assay: To perform the HIV drug susceptibility assay, R3/X4/R5 cell monolayers in 75 cm² flasks were treated with trypsin/versene to remove the cells from the flask. The cells were suspended in RPMI 1640 supplemented with 10% FBS and antibiotics. Then, 5×10⁴ cells were placed in each well of a 12 well plate and the plate was incubated at 37° C. under an atmosphere of 5% CO₂ for 18 hr. The medium was removed from the wells and 0.2 ml of virus in RPMI 1640 medium containing 10% FBS and 20 μg per ml of polybrene was added to each well. After a 2 hr adsorption period at 37° C. under 5% CO₂, the inoculum was removed and 1 ml of RPMI 1640 medium supplemented with 10% FBS, antibiotics and various concentrations of antiretroviral drugs were added to the appropriate wells. The plates were incubated at 37° C. under 5% CO₂ for 48 hr. The cell monolayers were treated with trypsin/versene to harvest the cells. The suspended cells were washed 2× with PBS, suspended in 1% formaldehyde and the percentage of cells expressing GFP was measured by flow cytometry. The flow cytometric analysis was of 10,000 cells. Initially, the cells were analyzed for forward versus right angle light scatter to distinguish between intact cells and debris. A gate was drawn around the population of cells that represented intact cells and the cells within the gate were analyzed for fluorescence intensity (Y axis) versus forward angle light scatter (X axis). A gate was drawn between the population of cells with low fluorescence intensity and the population of cells with higher fluorescence intensity. The cells above that gate represented the cells that expressed GFP as a result of productive HIV infection.

Statistics: The IC₅₀ values for each drug and each HIV isolates were determined by calculating the percent reduction in the number of GFP expressing cells at each drug concentration as compared with the number of GFP expressing cells in the absence of drug and plotting the percent reduction against the drug concentration.

Results

Table 1 lists the viruses used in these experiments. Most of the isolates are wild type (WT) and not resistant to any of the drugs used in these experiments. Clinical isolate A018-H112-2 was isolated from an AIDS patient in Africa before AZT treatment. Clinical isolate A018-G910-6 was isolated from the same African AIDS patient after 2 years of AZT treatment. HIV_{M184V} is a molecular clone of HIV_{LAI} with a mutation at 184 that leads to 3TC resistance. HIVJK_{NL43} is a macrophage tropic virus whereas all of the other viruses are lymphotropic viruses.

TABLE 1
Characteristics Of HIV Clinical Isolates

| | | | CO- | P 24 |
|---|---|---|---|---|
| CLINICAL | | | RECEPTOR | ANTIGEN |
| ISOLATE | SOURCE | PHENOTYPE | USAGE | ng/ml |
| HIV_{IIIB} | NIH | WT | R4 | 12.8 |
| HIV_{IIIB} | NIH | WT | R4 | ND |
| INFECTED | | | | |
| CELLS | | | | |
| A018-H11 | NIH | AZT | R4 | 500.2 |
| | | RESISTANT | | |
| A018-G910-6 | NIH | AZT | R4 | 11.1 |
| | | RESISTANT | | |
| HIV_{LAI}Bru | NIH | WT | R4 | 209.1 |
| HIV_{MI84V} | NIH | 3TC | R4 | 321 |
| | | RESISTANT | | |
| HIV 89.6 | NIH | WT | R4 | 221 |
| HIVJK_{NL43} | NIH | WT | R5 | ND¹ |
| 93000016 | ACTG | WT | R4 | 264 |
| 93000029 | ACTG | WT | R4 | 200 |
| 93000057 | ACTG | WT | R4 | 335 |

¹ND = NOT DETERMINED

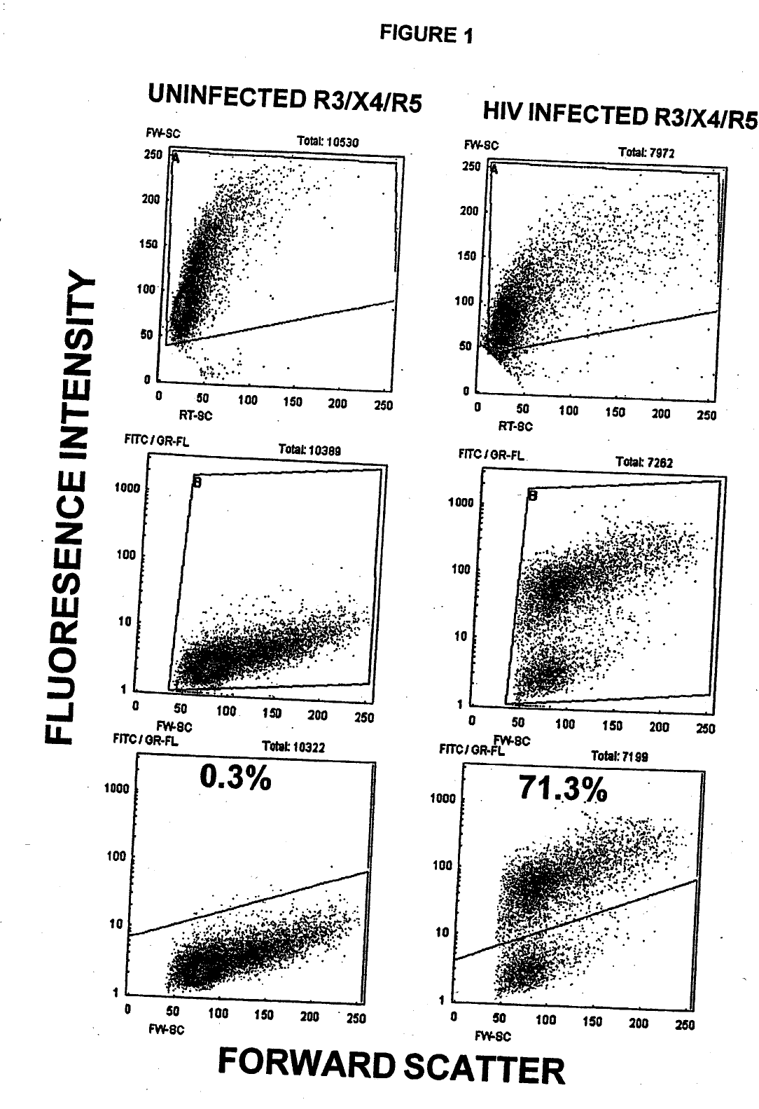

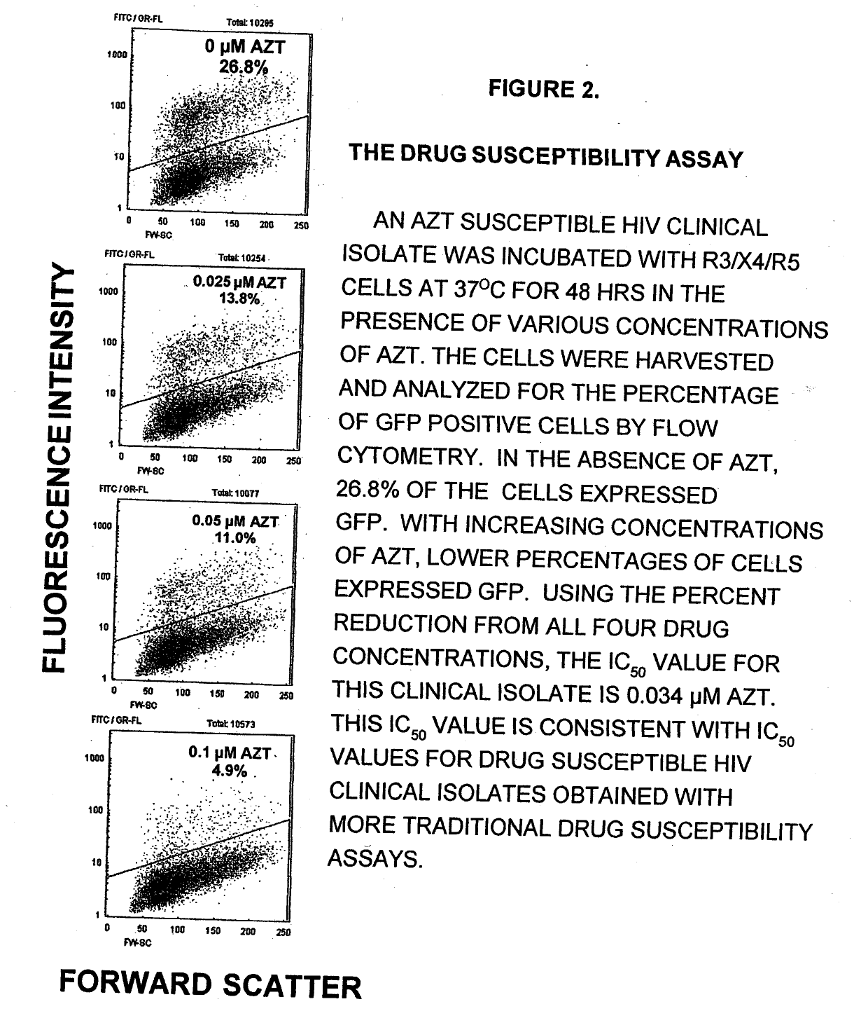

R3/X4/R5 cell monolayers were mock infected or infected with HIV. After two days of incubation, the cells were analyzed for the expression of GFP by flow cytometry, as shown in FIG. 1. The upper left and right hand panels illustrate forward and right angle light scatter analysis of uninfected and HIV infected R3/X4/R5 cells, respectively. Uninfected R3/X4/R5 cells scatter less light than the HIV infected R3/X4/R5 cells suggesting that the infected cells are larger and more granular than the uninfected cells. The middle panels show the analysis of fluorescence intensity versus forward light scatter for the cells identified by light scatter in the upper panels. Analysis of uninfected R3/X4/R5 cells shows one population of cells with low fluorescence intensity where as analysis of HIV infected R3/X4/R5 shows two populations of cells, one with low fluorescence intensity similar to the uninfected cells and the other with higher fluorescence intensity. In the lower panels, a gate is drawn above the cells with the low fluorescence intensity so that only 0.3% of the events are above the gate. When a gate is drawn for the HIV infected cells, 71.3% of the cells are above the gate suggesting that 71.3% of the cells are expressing GFP.

R3/X4/R5 cell monolayers were infected with an AZT susceptible HIV clinical isolate in the absence and presence of different concentrations of AZT. The effect of AZT on HIV replication was determined by measuring the number of cells expressing GFP by flow cytometry. In the absence of AZT, 26.8% of the cells expressed GFP. With increasing concentrations of AZT, the percent of GFP expressing cells declined. By calculating the percent reduction in percent GFP expressing cells at each drug concentration and plotting it against the drug concentration, the IC₅₀ for AZT for this clinical isolate was determined to be 0.034 μM. This value is consistent with IC₅₀ values for drug susceptible HIV clinical isolates determined by the more time consuming and costly phenotypic drug susceptibility assays.

Table 2 illustrates the effect of different antiviral drugs on infection of R3/X4/R5 cells with HIV_{IIIB}. R3/X4/R5 cell monolayers were infected with the HIV laboratory strain, HIV_{IIIB}, in the absence and presence of various concentrations of different antiviral drugs. The data show that treatment of uninfected cells with the highest concentration of each drug had no effect on the number of cells expressing GFP. The IC₅₀ value for Zidovudine for HIV_{IIIB} was 0.07 μM; for efavirenz for HIV_{IIIB} was 1.56 nM; and for amprenavir for HIV_{IIIB} was 10.78 nM. These values are consistent with IC₅₀ values for these drugs for HIV_{IIIB} obtained with more traditional phenotypic drug susceptibility assays.

TABLE 2
Effect Of Zidovudine On The Percentage Of Cells Expressing GFP

| | AZT | % GFP + | % | |
|---|---|---|---|---|
| CELLS | (μM) | CELLS | REDUCTION | IC₅₀ |
| R3/X4/R5 | 10 | 0.1 | — | |
| R3/X4/R5 + HIV | 0 | 29.6 | 0 | |
| R3/X4/R5 + HIV | 0.1 | 11.6 | 60.81 | 0.07 μM |
| R3/X4/R5 + HIV | 1.0 | 8.3 | 71.96 | |
| R3/X4/R5 + HIV | 10 | 0.4 | 98.65 | |

Effect Of Efavirenz On The Percentage Of Cells Expressing GFP

| | | % GFP + | % | |
|---|---|---|---|---|
| CELLS | EFA (nM) | CELLS | REDUCTION | IC₅₀ |
| R3/X4/R5 | 100 | 0.2 | — | |
| R3/X4/R5 + HIV | 0 | 35.0 | 0 | |
| R3/X4/R5 + HIV | 1 | 15.7 | 55.14 | 1.56 nM |
| R3/X4/R5 + HIV | 10 | 4.3 | 87.71 | |
| R3/X4/R5 + HIV | 100 | 0.8 | 97.71 | |

Effect Of Amprenavir On The Percentage Of Cells Expressing GFP

| | AMP | % GFP + | % | |
|---|---|---|---|---|
| CELLS | (nM) | CELLS | REDUCTION | IC₅₀ |
| R3/X4/R5 | 1000 | 0.1 | — | |
| R3/X4/R5 + HIV | 0 | 46.3 | — | |
| R3/X4/R5 + HIV | 10 | 25.5 | 45 | 10.78 nM |
| R3/X4/R5 + HIV | 100 | 1.1 | 98 | |
| R3/X4/R5 + HIV | 1000 | 0.1 | 99.8 | |

Table 3 illustrates that R3/X4/R5 cell monolayers can be used to determine IC₅₀ values for both lymphotropic and macrophage tropic viruses. Monolayers of R3/X4/R5 cells were infected with a lymphotropic strain of HIV, HIV_{IIIB}, or a macrophage tropic strain of HIV, HIVJK_{NL43}, in the absence and presence of various concentrations of AZT. The data show that the assay can be used to determine IC₅₀ values for this drug for both lymphotropic or macrophage tropic HIV laboratory strains.

TABLE 3

| | | |
|---|---|---|
| AZT (μM) | % GFP + CELLS | IC₅₀ |

Lymphotropic Virus (HIV_{IIIB}) Drug
Susceptibility Assay In R3/X4/R5 Cells

| | | |
|---|---|---|
| 0 | 71.4 | |
| 0.1 | 35.5 | 0.1 μM |
| 1.0 | 31.5 | |
| 10.0 | 29.6 | |

Macrophage Tropic Virus (JKNL43) Drug
Susceptibility Assay In R3/X4/R5 Cells

| | | |
|---|---|---|
| 0 | 33.1 | |
| 0.1 | 14.9 | 0.09 μM |
| 1.0 | 17.1 | |
| 10.0 | 11.1 | |

Table 4 illustrates the effect of AZT on clinical isolates. HIV_{IIIB} and three clinical isolates obtained from the Albany Medical College ACTG were incubated with R3/X4/R5 cell monolayers in the presence or absence of various concentrations of AZT. After 48 hr, the cells were harvested and assayed for the expression of GFP by flow cytometry. The data show that AZT reduced the number of cells expressing GFP after infection with each clinical isolate. IC₅₀ values for AZT for these four drug susceptible HIV samples were similar. The low IC₅₀ values indicate that each clinical isolate is susceptible to AZT. These results show that the assay can be used for HIV clinical isolates as well as HIV laboratory strains.

TABLE 4
Effect Of AZT On R3/X4/R5 Cells Infected With HIV Clinical Isolates

| ISOLATE | AZT (μM) | % GFP + CELLS | IC₅₀ |
|---|---|---|---|
| HIV_{IIIB} | 0 | 13.8 | |
| | 0.1 | 0.8 | 0.04 μM |
| | 1.0 | 0.7 | |
| | 10 | 0.5 | |
| 93000016 | 0 | 84.4 | |
| 3.3 × 10¹⁰ COPIES/ml | 0.1 | 7.1 | 0.06 μM |
| | 1.0 | 1.6 | |
| | 10.0 | 1.6 | |
| 93000029 | 0 | 34.1 | |
| 3.5 × 10¹⁰ COPIES/ml | 0.1 | 1.2 | 0.04 μM |
| | 1.0 | 0.6 | |
| | 10 | 1.1 | |
| 93000057 | 0 | 10.7 | |
| 1.2 × 10¹⁰ COPIES/ml | 0.1 | 2.6 | 0.07 μM |
| | 1.0 | 0.7 | |
| | 10.0 | 0.7 | |

Table 5 shows the IC₅₀ values for drug susceptible and drug resistant HIV isolates to four different drugs. Various HIV isolates were incubated with R3/X4/R5 cell monolayers in the absence and presence of various antiretroviral drugs. The data in Table 5 show that the HIV clinical isolate, 93000016, had low IC₅₀, indicating sensitivity to AZT, 3TC, DLV, and ABC. The IC₅₀ values for the pre-AZT clinical isolate, A018-H112-2, indicated sensitivity to AZT, 3TC, DLV and ABC, but not as sensitive to AZT as clinical isolate 93000016. The post-AZT clinical isolate was resistant to AZT and sensitive to 3TC, DLV, and ABC. The post AZT clinical isolate was not as sensitive as the pre-AZT isolate or clinical isolate 93000016 to 3TC. HIV_{M184V} was sensitive to AZT, resistant to 3TC, and sensitive to DLV and ABC. HIV_{Bru} and HIV 89.6 were sensitive to all four drugs. These results show that the assay can be used to evaluate the susceptibilities of both drug susceptible and drug resistant HIV clinical isolates for a number of different antiretroviral drugs.

TABLE 5
Average IC₅₀ Values Of Drugs For HIV Clinical Samples

| HIV ISOLATE | AZT (μM) | 3TC (μM) | DLV (μM) | ABC (μM) |
|---|---|---|---|---|
| 93000016 | 0.06 ± 0.04 | 1.76 ± 0.009 | 0.05 ± 0.02 | 1.60 ± 0.06 |
| A018-H112-2 | 0.19 ± 0.02 | 1.73 ± 0.09 | 0.04 ± 0.01 | 2.52 ± 0.58 |
| A018-G910-6 | >20 | 5.28 ± 0.81 | 0.05 ± 0.02 | 2.25 ± 0.20 |
| HIV_{M184V} | 0.04 ± 0.01 | >20 | 0.04 ± 0.005 | 2.20 ± 0.33 |
| H1V_{Bru} | 0.04 ± 0.005 | 1.02 ± 0.13 | 0.03 ± 0.005 | 1.16 ± 0.39 |
| HIV 89.6 | 0.03 ± 0.009 | 2.24 ± 0.60 | 0.03 ± 0.005 | 1.66 ± 0.54 |

Each IC₅₀ value is an average of four determinations
AZT—Zidovudine
3TC—Lamivudine
DLV—Delavirdine
ABC—Abacavir

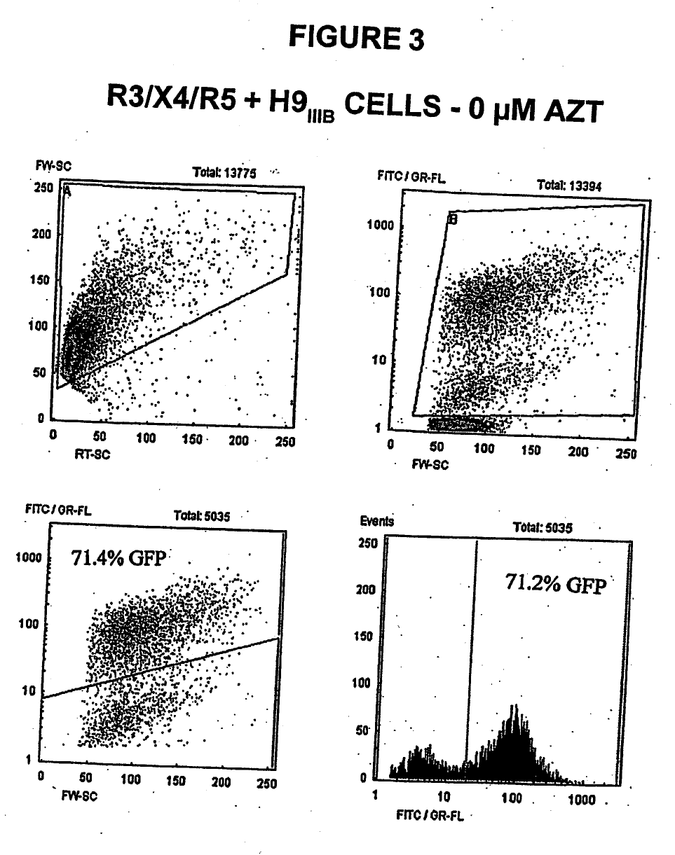

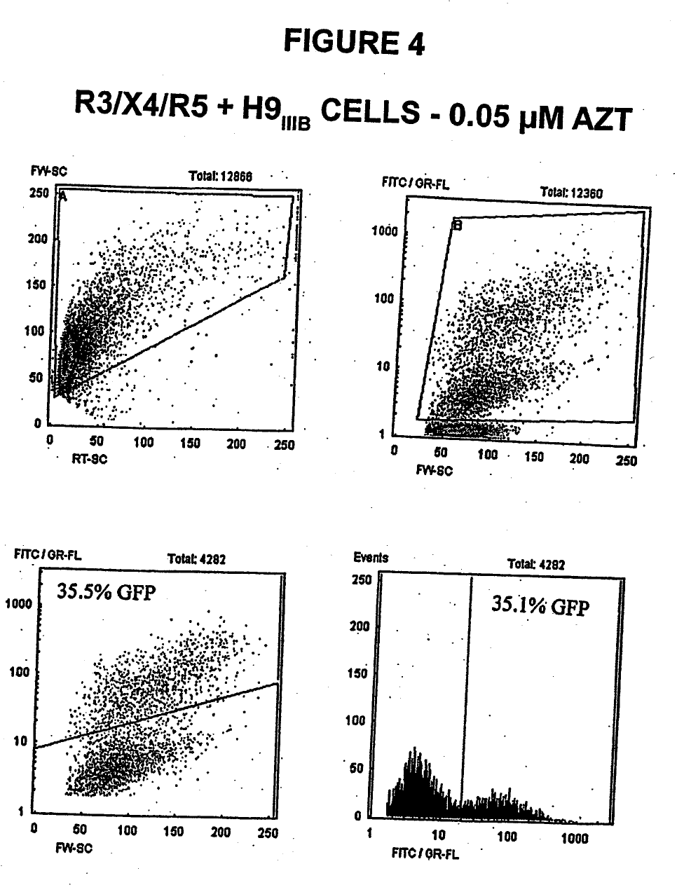

FIGS. 3 and 4 show the use of virus infected cells in a drug susceptibility assay of the invention. R3/X4/R5 cell monolayers were infected with H9 cells chronically infected with HIV_{IIIB} in the absence (FIG. 3) and in the presence of AZT (FIG. 4). After 48 hr of incubation the number of cells expressing GFP was determined by flow cytometry. In the absence of AZT, 71.4% of the cells express GFP, whereas in the presence of 0.05 μM AZT only 35.1% of the cells express GFP. These results show that cell associated HIV can be used in the assay to determine the susceptibility of HIV to AZT. The results of this experiment showing that cell associated virus can be used in the drug susceptibility assay opens the way to do drug susceptibility testing directly on clinical samples obtained from HIV infected individuals. The ability to test virus for susceptibility to drugs without first growing the virus will save considerable time. This will yield a clinically useful assay for managing HIV infected patients.

### EXAMPLE 2

In use of a drug susceptibility assay of the invention for HIV clinical isolates, after obtaining informed consent from each patient, 20 ml of peripheral blood will be collected by venapuncture and the plasma will be separated from the white and red blood cells by centrifugation in Accuspin histopague tubes (Sigma) for 15 minutes at 1800 rpm. The plasma will be removed and frozen at −70° C. The band of leukocytes cells will be aspirated from the interface with histopague, washed 2× in Hanks balanced salt solution and suspended in RPMI 1640 medium containing 10% heat inactivated fetal bovine serum (ΔFBS). The cells will be cultured in RPMI1640 medium and 10% ΔFBS in the presence of IL-2 and PHA for 3 days to activate the latent virus in the CD4⁺ T cells and then the activated cells will be co-cultured with R3/X4/R5 cells in the presence of various concentrations of antiretroviral drugs for 48 hr at 37° C. Then, the cells will be removed from the plates and the number of cells expressing GFP will be determined by flow cytometry.

Cell free HIV in the plasma will be concentrated using polyethylene glycol precipitation (McSharry and Benzinger, 1970, Virology 40:745-746; Fiscus et al, 1991 J Infect. Dis. 164:765-769; Fiscus et al., 1993. Viral Immunology 6:135-141), the virus in the precipitate will be suspended in RPMI 1640 medium with 10% ΔFBS and added to monolayers of R3/X4/R5 cells. After 48 hr of incubation, the cells will be harvested and the number of cells expressing GFP will be determined by flow cytometry.

For either cell free or cell associated HIV, the percent reduction in the number of GFP cells will be plotted against the drug concentration to determine the IC₅₀ values. Each assay will be performed in triplicate.

Equivalents

Those skilled in the art will recognize, or be able to ascertain using no more than routine experimentation, numerous equivalents to the specific procedures described herein. Such equivalents are considered to be within the scope of the present invention and are covered by the following claims. The contents of all references, issued patents, and published patent applications cited throughout this application are hereby incorporated by reference. The appropriate components, processes, and methods of those patents, applications and other documents may be selected for the present invention and embodiments thereof.

What is claimed is:

1. An assay for determining the efficacy of candidate HIV inhibitors, comprising
a) infecting a cell line with HIV in the presence of a putative HIV inhibitor, said cell line expressing CD4, CXCR4 and CCR5 receptors on the cell surface, and a marker gene product; wherein said marker gene product expression increases in response to said cell line infection with HIV; and
b) dynamically counting the number of said cells expressing said marker gene product and comparing the number of cells expressing said marker gene product to a control value to determine whether said putative HIV inhibitor is an inhibitor of HIV.

2. The assay of claim 1, wherein said marker gene product is selected from the group consisting of chloramphenicol acetyltransferase (CAT), β-galactosidase (GAL), β-glucuronidase (GUS), luciferases (LUC), ferredoxin IV, green fluorescent protein, red fluorescent protein, yellow fluorescent protein, blue fluorescent protein, and the aequorin family.

3. The assay of claim 1, wherein said marker gene product is a cell-associated marker composition.

4. The assay of claim 1, wherein said marker gene product is a luminescent protein.

5. The assay of claim 1, wherein said luminescent protein is selected from the group consisting of GFP and luciferase.

6. The assay of claim 1, wherein said cell line further expresses one or more receptors selected from the group consisting of CCR1, CCR2b, CCR3, CCR4, CCR8, CXCR1, CXCR2, CXCR3, CX, CR1, STRL22/BONZO and GPR15/BOB.

7. The assay of claim 1, wherein said cells are osteosarcoma cells.

8. The assay of claim 1, wherein said dynamic counting of said cells is done by flow cytometry.

9. The assay of claim 1, wherein said putative HIV inhibitor affects events in the HIV replication cycle selected from the group consisting of protease inhibition, attachment, uncoating, reverse transcription, and integration steps.

10. The assay of claim 1, wherein said HIV is selected from the group consisting of lymphotrophic, macrophage trophic, and dual trophic strains.

11. The assay of claim 1, wherein said HIV is a cell free virus.

12. The assay of claim 1, wherein said HIV is in a virus infected cell.

13. The assay of claim 1, wherein said HIV is in a clinical isolate.

14. A method for screening clinical isolates for determining the efficacy of candidate HIV inhibitors, comprising
a) contacting a cell line with a clinical isolate suspected of containing HIV in the presence of a putative HIV inhibitor, said cell line expressing CD4, CXCR4 and CCR5 receptors on the cell surface, and a marker gene product; wherein said marker gene product expression increases in response to cell line infection with HIV; and
b) dynamically counting the number of said cells expressing said marker gene product and comparing the number of cells expressing said marker gene product to a control value to determine whether said putative HIV inhibitor is an inhibitor of HIV.

15. The method of claim 14, wherein said marker gene product is selected from the group consisting of chloramphenicol acetyltransferase (CAT), β-galactosidase (GAL), β-glucuronidase (GUS), luciferases (LUC), ferredoxin IV, green fluorescent protein, red fluorescent protein, yellow fluorescent protein, blue fluorescent protein, and the aequorin family.

16. The method of claim 14, wherein said marker gene product is a cell-associated marker composition.

17. The method of claim 14, wherein said marker gene product is a luminescent protein.

18. The method of claim 14, wherein said luminescent protein is selected from the group consisting of GFP and luciferase.

19. The method of claim 14, wherein said cell line further expresses one or more receptors selected from the group consisting of CCR1, CCR2b, CCR3, CCR4, CCR8, CXCR1, CXCR2, CXCR3, CX, CR1, STRL22/BONZO and GPR15/BOB.

20. The method of claim 14, wherein said cells are osteosarcoma cells.

21. The method of claim 14, wherein said dynamic counting of said cells is done by flow cytometry.

22. The method of claim 14, wherein said putative HIV inhibitor affects events in the HIV replication cycle selected from the group consisting of attachment, uncoating, reverse transcription, and integration steps.

23. The method of claim 14, wherein said HIV is selected from the group consisting of lymphotrophic, macrophage trophic, and dual trophic strains.

24. The method of claim 14, wherein said HIV is a cell free virus.

25. The method of claim 14, wherein said HIV is in a virus infected cell.
